(19) United States
(12) Patent Application Publication
(10) Pub. No.: US 2015/0044752 A1
(43) Pub. Date: Feb. 12, 2015

(54) Method of Treatment of Mesenchymal Stem Cells

(71) Applicant: Lee M. Buono, Medford, NJ (US)

(72) Inventor: Lee M. Buono, Medford, NJ (US)

(21) Appl. No.: 14/451,477

(22) Filed: Aug. 5, 2014

Related U.S. Application Data
(60) Provisional application No. 61/863,955, filed on Aug. 9, 2013.

(51) Int. Cl. C12N 13/00; C12N 5/0775

(52) U.S. Cl. CPC C12N 13/00; C12N 5/0675
USPC 435/173.8

(57) ABSTRACT

Extracted human mesenchymal cells (MSCs) are placed into the interior space of a closed vessel and exposed to an electric AC current and/or electromagnetic field. The MSCs are also stirred within the vessel to further stimulate the cells, enhancing the osteodifferentiation process during human transplantation of the cells.

### RELATED APPLICATIONS

The herein application claims the benefit of provisional application Ser. No. 61/863,955, filed Aug. 9, 2013.

### BACKGROUND OF THE INVENTION

Human mesenchymel stem cells (MSCs) are self-renewing adult cells, normally isolated in the bone marrow, but also found in other tissues such as cord blood, peripheral blood, fallopian tube, and fetal liver and lungs. MSCs are “multipotent”, in that they can produce more than one, but not all types of specialized cells of the body. MSCs can differentiate, a process whereby a less specialized cell becomes a more specialized cell type. MSCs differentiate into osteoblasts (bone cells), chondrocytes (cartilage cells), and adipocytes (fat cells). Osteodifferentiation is the differentiation of MSCs into bone tissue.

### SUMMARY OF THE INVENTION

The method of the present invention relates to affecting MSCs in order to enhance human transplantation. It has been found that upon exposure of MSCs to alternating electric current (AC) and/or electromagnetic field during the human transplantation process, stimulates osteodifferentiation, when compared to controlled stem cell treatments.

The application of AC current and/or electromagnetic field directly to these MSCs prior to implantation at the human graft site is significant to the stimulation of the stem cells for differentiating into osteoblasts. The subsequent stirring of the cells provides them with maximum exposure to the AC current and/or electromagnetic, field prior to implantation.

The novel features which are considered as characteristics of the invention set forth in particular in the appended claims. The invention, itself, however, both as to its design, construction and use, together with additional features and advantages thereof, are best understood upon review of the following detailed description with reference to the accompanying drawings.

### BRIEF DESCRIPTION OF THE DRAWINGS

FIG. 1 is an elevation view of the closed vessel in which MSCs are exposed.

FIG. 2 is a top view of the closed vessel shown in FIG. 1, with the cap of the vessel removed depicting the stimulation of MSCs.

### DETAILED DESCRIPTION OF THE INVENTION

Extracted human MSCs are placed into the interior space of a closed vessel exposed to an electric AC current and/or electromagnetic field. Studies suggest that integral to the inventive method, the application of AC current of 10-40 microamps or electromagnetic field of 20 mV/cm, 60 kHz to MSCs for a period of 10 to 15 minutes stimulates the desired osteodifferentiation of the cells. During the exposure within the closed vessel, the MSCs are stirred using a sterile stirrer, which could be free floating within the vessel in which the cells are placed. Once simultaneous AC electric current and/or electromagnetic field stimulation and stirring are complete, MSCs are available for transplant to the human graft site.

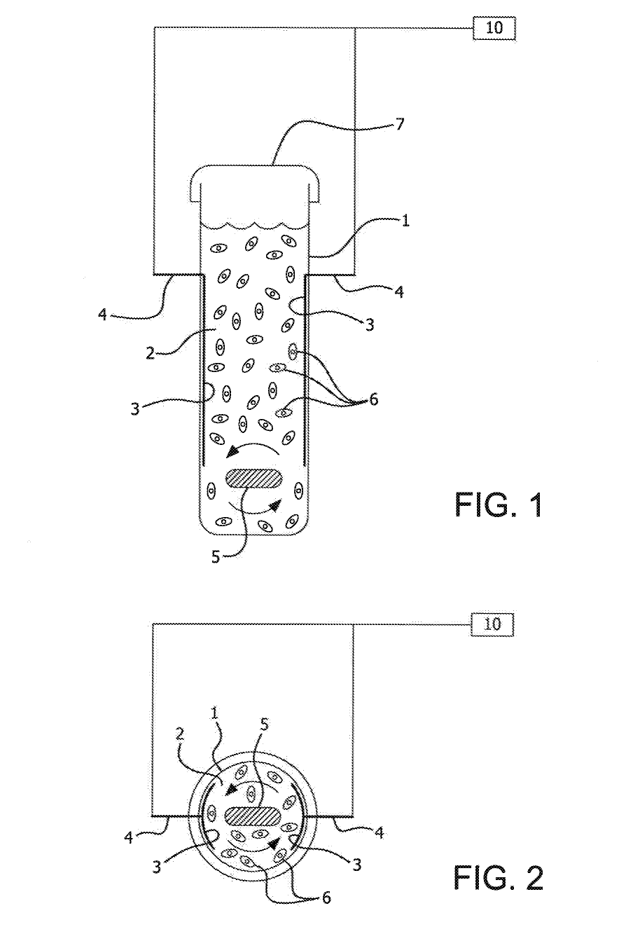

FIG. 1 shows vessel 1 having interior space 2. Electrode plates 3 with conductive metal contacts 4 are mounted along the exterior surface of the vessel. A plurality of MSCs are placed within interior space 2 of vessel 1 and the vessel s sealed by watertight cap 7. AC electric current and/or electromagnetic field from source 10 is delivered to conductive metal contacts 4 and then to electrodes 3. The AC current and/or electromagnetic field is passed from electrodes 3 into interior space 2 of vessel 1, to affect and electrically charge MSCs 6 within the interior space. Free floating plastic coated metal stirrer 5, located within interior space 2, stirs electrically charged MSCs 6. This provides MSCs with maximum exposure to the electric current and/or electromagnetic field.

After a period of time ranging from 10 to 15 minutes, MSCs 6 are removed from vessel 1 transplantation.

Thus, the present invention describes a unique method for the stimulation of MSCs. The invention should not be considered restricted to the use of the vessel, stirrer or the electric current or electromagnetic power source which are specifically disclosed, as it is contemplated that equivalent components can be utilized to effect the method.

Certain novel features and components of this invention are disclosed in detail in order to make the invention clear in at least one form thereof. However, it is to be clearly understood that the invention as disclosed is not necessarily limited to the exact form and details as disclosed, since it is apparent that various modification and changes may he made without departing from the spirit of the invention.

1. A method for treating mesenchymal stem cells (MSCs) prior to differentiating the cells for human transplantation, the steps comprising:
providing a vessel having an interior space and an exterior surface on which electrode plates while mounted;
providing a plurality of MSCs;
placing the plurality of MSCs within the interior space of the vessel;
delivering electric current to the electrode plates;
delivering said electric current to the interior space of the vessel and to the MSCs located in the interior space, thus electrically charging the MSCs;
stirring the electrically charged MSCs within the interior space; and
removing the MSCs from the vessel for transplantation.

2. The method as in claim 1 further comprising the step of providing a cover on the vessel for ensuring for a watertight seal over the vessel.

3. The method as in claim 1 further comprising the step of providing a free floating stirrer within the interior space of the vessel for stirring the electrically charged MSCs.

4. The method as in claim 1 comprising the further step of delivering the electric current to the electrode plates and thus the MSCs and stirring the MSCs within the vessel for a period of time ranging between 10 and 15 minutes.

5. A method for treating mesenchymal stem cells (MSCs) prior to differentiating the cells for human transplantation the steps comprising:
providing a vessel having an interior space and an exterior surface on which electrode plates while mounted;
providing a plurality of MSCs;
placing the plurality of MSCs within the interior space of the vessel;
delivering an electromagnetic field to the electrode plates.
delivering said electromagnetic field to the interior space of the vessel and to the MSCs located in the interior space, thus electrically charging the MSCs;
stirring the electrically charged MSCs within the interior space; and
removing the MSCs from the vessel for transplantation.

6. The method as in claim 5 further comprising the step of providing a cover on the vessel for ensuring fur a watertight seal over the vessel.

7. The method as in claim 5 further comprising the step of providing a free floating stirrer within the interior space of the vessel for stirring the electrically charged MSCs.

8. The method as in claim 5 comprising the further step of delivering the electromagnetic field to the electrode plates and thus electrically charging the MSCs and stirring the MSCs within the vessel for a period of time ranging between 10 and 15 minutes.
